(19) United States
(12) Patent Application Publication
(10) Pub. No.: US 2026/0158253 A1
(43) Pub. Date: Jun. 11, 2026

(54) DRUG INJECTION DEVICE

(71) Applicant: SEOUL NATIONAL UNIVERSITY R&DB FOUNDATION, Seoul (KR)

(72) Inventor: SUNG JOON YE, Seoul (KR)

(21) Appl. No.: 18/709,889

(22) PCT Filed: Mar. 23, 2023
(86) PCT No.: PCT/KR2023/003843

§ 371 Date: May 14, 2024

(30) Foreign Application Priority Data
Apr. 6, 2022 (KR) 10-2022-0043037
Feb. 7, 2023 (KR) 10-2023-0016007

(51) Int. Cl. A61M 37/00

(52) U.S. Cl. CPC A61M 37/0015; A61M 2037/0023; A61M 2037/003; A61M 2037/0046

(57) ABSTRACT

A drug delivery device includes a lumen unit to form an internal channel and an external channel, a gas unit to supply a gas to the internal channel, and a drug solution unit to supply a drug solution to the external channel. The lumen unit includes an inner coating including first and second inner surfaces, and micropores passing through the first and second inner surfaces, an outer coating including a first outer surface facing the second inner surface, a second outer surface positioned opposite the first outer surface, and a microneedle plate on the second outer surface, the internal channel surrounded by the first inner surface, and the external channel between the second inner surface and the first outer surface. The gas and the drug solution are mixed in the external channel to form a mixed drug solution, which is supplied to an outside through the microneedle plate.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2023/003843 filed on Mar. 23, 2023, which claims priority to the benefit of Korean Patent Application Nos. 10-2022-0043037 filed on Apr. 6, 2022 and 10-2023-0016007 filed on Feb. 7, 2023 in the Korean Intellectual Property Office, the entire contents of which are incorporated herein by reference.

### BACKGROUND

### 1. Technical Field

The present disclosure relates to a drug delivery device. More particularly, the present disclosure relates to a disease site targeting drug delivery device delivering a mixed drug solution, which is a mixture of a drug solution and oxygen, directly to an affected area.

### 2. Background Art

Currently, a most widely used method of delivering anticancer drugs is to simply dissolve non-water soluble anticancer drugs in an organic solvent and inject it directly. This method may deliver the anticancer drugs to normal cells as well as cancer cells and cause many side effects due to the toxicity of the organic solvent.

To overcome the above-mentioned side effects, a method of delivering drugs directly to the affected area was developed. The direct drug delivery method refers to a method in which the drugs pass through the epidermal layer of the affected area and are delivered directly to the inside of the affected area such as the dermal layer. Because the drugs are target-delivered directly to the local area, this method is attracting attention as a more effective treatment method than an injection method.

As the direct drug delivery method, a drug delivery device based on a microneedle array that enables targeting delivery of drugs directly to the local area is being introduced. The microneedle array may allow the drugs to pass through a surface layer or an upper layer of the affected area and to be target-delivered to the affected area, in order to increase percutaneous drug penetration. However, the above-described method has a limited capability to deliver drugs or vaccines only to the epidermal layer of the affected area.

As described above, because the existing drug delivery device simply has only a drug delivery capability, it is difficult to maximize the enhanced permeability and retention (EPR) effect in which nanoparticles are accumulated in tumor tissue.

Accordingly, there is a need for a drug delivery device that can overcome the practical limitation of the EPR effect which is the core of nanoparticle anti-cancer treatment (i.e., drug delivery principle) using a difference between cancer blood vessel walls and can push nanoparticle drugs deep into the affected area.

### SUMMARY

An object of the present disclosure is to address the above-described and other problems.

Another object of the present disclosure is to provide a drug delivery device capable of pushing a nanoparticle drug deep into an affected area.

Another object of the present disclosure is to provide a drug delivery device that can overcome hypoxia with a mixed drug solution which is a mixture of a nanoparticle drug and micro-oxygen droplets.

In order to achieve the above-described and other objects and needs, according to one aspect of the present disclosure, a drug delivery device may comprise a lumen unit configured to form an internal channel and an external channel, a gas unit configured to supply a gas to the internal channel, and a drug solution unit configured to supply a drug solution to the external channel.

The lumen unit may include an inner coating including a first inner surface, a second inner surface positioned opposite the first inner surface, and micropores passing through the first inner surface and the second inner surface, an outer coating including a first outer surface facing the second inner surface, a second outer surface positioned opposite the first outer surface, and a microneedle plate formed on a surface of the second outer surface, the internal channel surrounded by the first inner surface, and the external channel formed between the second inner surface and the first outer surface.

The gas and the drug solution may be mixed in the external channel to form a mixed drug solution, and the mixed drug solution may be supplied to an outside through the microneedle plate.

The internal channel and the external channel may communicate with each other through the micropores.

The micropores may allow the gas to pass through from the internal channel to the external channel and prevent the drug solution from passing through from the external channel to the internal channel.

The inner coating may be formed of at least one of polyurethane resin, Teflon resin, polycaprolactone (PCL) nanofiber membrane, and mixtures thereof.

The outer coating may include polyester, polyhydroxy alkanoate (PHAs), poly (α-hydroxy acid), poly(β-hydroxy acid), poly(3-hydroxybutyrate-co-valerate) (PHBV), poly(3-hydroxypropionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), polycaprolactone, polylactide, polyglycolide, poly(lactide-co-glycolide) (PLGA), polydioxanone, polyorthoester, polyether ester, polyanhydride, poly(glycolic acid-co-trimethylene carbonate), polyphosphoester, polyphosphoester urethane, poly(amino acid), polycyanoacrylate, poly(trimethylene carbonate), poly(imino carbonate), poly(tyrosine carbonate), polycarbonate, poly(tyrosine arylate), polyalkylene oxalate, polyphosphazenes, PHA-PEG, ethylene vinyl alcohol copolymer (EVOH), polyurethane, silicone, polyester, polyolefin, polyisobutylene and ethylene-alpha olefin copolymer, styrene-isobutylene-styrene triblock copolymer, acrylic polymer and copolymer, vinyl halide polymer and copolymer, polyvinyl chloride, polyvinyl ether, polyvinyl methyl ether, polyvinylidene halide, polyvinylidene fluoride, polyvinylidene chloride, polyfluoroalkene, polyperfluoroalkene, polyacrylonitrile, polyvinyl ketones, polyvinyl aromatics, polystyrene, polyvinyl ester, polyvinyl acetate, ethylene-methyl methacrylate copolymer, acrylonitrile-styrene copolymer, ABS resin and ethylene-vinyl acetate copolymer, polyamide, alkyd resin, polyoxymethylene, polyimide, polyether, polyacrylate, polymethacrylate, polyacrylic acid-co-maleic acid, chitosan, dextran, cellulose, heparin, hyaluronic acid, alginate, inulin, starch, or glycogen. The outer coating may be specifically formed of at least one selected among polyester, polyhydroxyalkanoate (PHAs), poly(α-hydroxy acid), poly(β-hydroxy acid), poly(3-hydroxy butyrate-co-valerate) (PHBV), poly(3-hydroxypropionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), polycaprolactone, polylactide, polyglycolide, poly(lactide-co-glycolide) (PLGA), polydioxanone, polyorthoester, polyether ester, polyanhydride, poly(glycolic acid-co-trimethylene carbonate), polyphosphoester, polyphosphoester urethane, poly(amino acid), polycyanoacrylate, poly(trimethylene carbonate), poly(imino carbonate), poly(tyrosine carbonate), polycarbonate, poly (tyrosine arylate), polyalkylene oxalate, polyphosphazenes, PHA-PEG, chitosan, dextran, cellulose, heparin, hyaluronic acid, alginate, inulin, starch, or glycogen.

The lumen unit may include a discharge area in which a spout part discharging the gas or the drug solution is disposed, a delivery area in which a delivery part delivering the gas or the drug solution is disposed, and an injection area in which a filling part filled with the gas or the drug solution is disposed.

The lumen unit may further include a first lumen unit formed of the inner coating and a second lumen unit formed of the outer coating. The first lumen unit and the second lumen unit may be disposed in the discharge area, the delivery area, and the injection area.

The first lumen unit may include a first spout part configured to discharge the gas and disposed in the discharge area, a first delivery part configured to deliver the gas and disposed in the delivery area, and a first filling part filled with the gas and disposed in the injection area.

The second lumen unit may include a second spout part configured to discharge the drug solution and disposed in the discharge area, a second delivery part configured to deliver the drug solution and disposed in the delivery area, and a second filling part filled with the drug solution and disposed in the injection area.

The microneedle plate may be disposed on the second filling part disposed in the injection area and may be formed by protruding a portion of a surface of the outer coating.

The microneedle plate may include a plurality of microneedles, and a plurality of microholes configured to communicate the external channel with the outside, at least one microhole being disposed in each of the plurality of microneedles.

The microneedle plate may push the plurality of microneedles into an inside of an affected area and bring a surface of the outer coating into close contact with a surface of the affected area.

An extension channel extending from the external channel may be formed inside the microneedle.

The microholes may communicate the external channel with the outside.

The microholes may include at least one of a first microhole in which a tip of the microneedle is formed at a vertex of a sharp end, a second microhole formed in a bent area in which the outer coating is bent to form the microneedle, and a third microhole formed in a side of the microneedle disposed between the vertex and the bent area.

A size of each of the micropores may be 1 nm to 50 nm.

When the gas is an oxygen gas, the gas may pass through the micropores and may be converted into micro-oxygen droplets.

A pressure of the gas provided to the internal channel may be 1 atm to 5 atm.

The drug solution may be a nanoparticle composite that is a mixture of a solvent and nanoparticles.

A particle size of the nanoparticle composite may be 50 nm to 200 nm.

The lumen unit may be flexible and elastic.

An amount of the drug solution and a pressure of the gas may be controlled by a monitoring sensor installed at an inlet of a double lumen.

Effects of a drug delivery device according to the present disclosure are described as follows.

According to at least one aspect of the present disclosure, the drug delivery device according to the present disclosure can push a nanoparticle drug deep into an affected area.

According to at least one aspect of the present disclosure, the drug delivery device according to the present disclosure can overcome hypoxia with a mixed drug solution which is a mixture of a nanoparticle drug and micro-oxygen droplets.

Additional scope of applicability of the present disclosure will become apparent from the detailed description given blow. However, it should be understood that the detailed description and specific examples such as embodiments of the present disclosure are given merely by way of example, since various changes and modifications within the spirit and scope of the present disclosure will become apparent to those skilled in the art from the detailed description.

### BRIEF DESCRIPTION OF THE DRAWINGS

FIG. 1 is a cross-sectional view of a drug delivery device according to an embodiment of the present disclosure.

FIG. 2 is a cross-sectional view taken along A1-A2 of FIG. 1.

FIG. 3 is a cross-sectional view taken along B1-B2 of FIG. 1.

FIG. 4 is a partial perspective view of an outer coating according to an embodiment of the present disclosure.

FIG. 5 is a cross-sectional view taken along C1-C2 of FIG. 4.

FIG. 6 is a cross-sectional view of a microneedle plate according to another embodiment of the present disclosure.

FIG. 7 is an enlarged cross-sectional view of a portion “Q” of FIG. 1.

FIG. 8 illustrates a drug delivery device including a flat lumen unit.

### DETAILED DESCRIPTION

Reference will now be made in detail to embodiments of the disclosure, examples of which are illustrated in the accompanying drawings. Wherever possible, the same reference numbers will be used throughout the drawings to refer to the same or like parts. In general, a suffix such as “module” and “unit” may be used to refer to elements or components. Use of such a suffix herein is merely intended to facilitate description of the present disclosure, and the suffix itself is not intended to give any special meaning or function. It will be noted that a detailed description of known arts will be omitted if it is determined that the detailed description of the known arts can obscure embodiments of the present disclosure. The accompanying drawings are used to help easily understand various technical features and it should be understood that embodiments presented herein are not limited by the accompanying drawings. As such, the present disclosure should be construed to extend to any alterations, equivalents and substitutes in addition to those which are particularly set out in the accompanying drawings.

The terms including an ordinal number such as first, second, etc. may be used to describe various components, but the components are not limited by such terms. The terms are used only for the purpose of distinguishing one component from other components.

When any component is described as “being connected” or “being coupled” to other component, this should be understood to mean that another component may exist between them, although any component may be directly connected or coupled to the other component. In contrast, when any component is described as “being directly connected” or “being directly coupled” to other component, this should be understood to mean that no component exists between them.

A singular expression can include a plural expression as long as it does not have an apparently different meaning in context.

In the present disclosure, terms “include” and “have” should be understood to be intended to designate that illustrated features, numbers, steps, operations, components, parts or combinations thereof are present and not to preclude the existence of one or more different features, numbers, steps, operations, components, parts or combinations thereof, or the possibility of the addition thereof.

In the drawings, sizes of the components may be exaggerated or reduced for convenience of explanation. For example, the size and the thickness of each component illustrated in the drawings are arbitrarily illustrated for convenience of explanation, and thus the present disclosure is not limited thereto unless specified as such.

If any embodiment is implementable differently, a specific order of processes may be performed differently from the order described. For example, two consecutively described processes may be performed substantially at the same time, or performed in the order opposite to the described order.

In the following embodiments, when layers, areas, components, etc. are connected, the following embodiments include both the case where layers, areas, and components are directly connected, and the case where layers, areas, and components are indirectly connected to other layers, areas, and components intervening between them. For example, when layers, areas, components, etc. are electrically connected, the present disclosure includes both the case where layers, areas, and components are directly electrically connected, and the case where layers, areas, and components are indirectly electrically connected to other layers, areas, and components intervening between them.

Reference will now be made in detail to embodiments of the present disclosure with reference to the accompanying drawings.

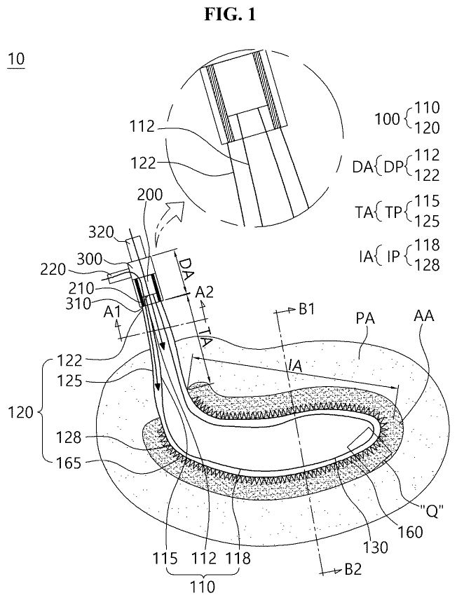

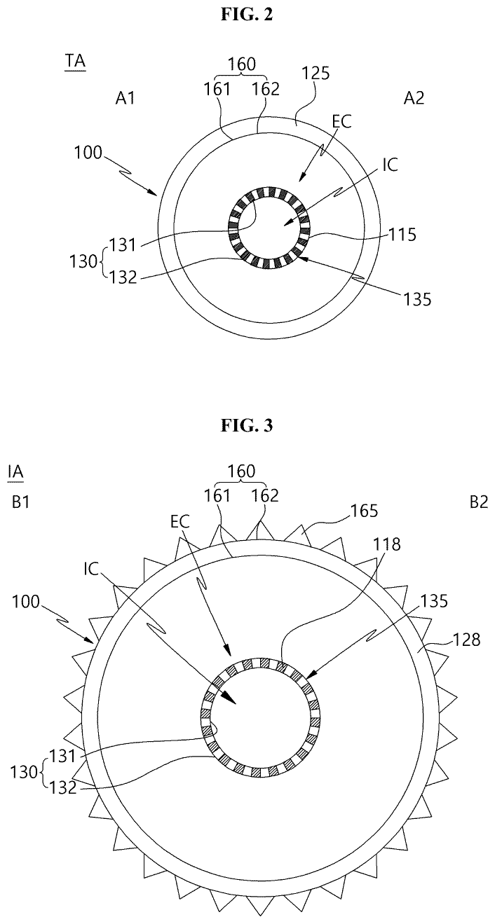

FIG. 1 is a cross-sectional view of a drug delivery device according to an embodiment of the present disclosure. FIG. 2 is a cross-sectional view taken along A1-A2 of FIG. 1. FIG. 3 is a cross-sectional view taken along B1-B2 of FIG. 1.

Referring to FIGS. 1 to 3, a drug delivery device 10 may include a lumen unit 100, a gas unit 200, and a drug solution unit 300.

The lumen unit 100 may include a discharge area DA, a delivery area TA, and an injection area IA. The lumen unit 100 may supply a gas provided from the gas unit 200 and a drug solution provided from the drug solution unit 300 to the outside. If the outside is an affected area AA of a patient, a drug may be target-delivered to the affected area AA. The affected area AA may be disposed in a partial area of a normal tissue PA.

The gas unit 200 may include a gas supply source (not shown) that supplies the gas, a gas supply pipe 220 that delivers the gas, and a gas discharge port 210 that discharges the gas. The gas supply pipe 220 may be disposed between the gas supply source and the gas discharge port 210. The gas discharge port 210 may be disposed in the discharge area DA.

The drug solution unit 300 may include a drug solution supply source that supplies a drug solution, a drug solution supply pipe 320 that delivers the drug solution, and a drug solution discharge port 310 that discharges the drug solution. The drug solution supply pipe 320 may be disposed between the drug solution supply source and the drug solution discharge port 310. The drug solution discharge port 310 may be disposed in the discharge area DA.

The lumen unit 100 may form a balloon shape. The gas unit 200 may provide the gas to the lumen unit 100. When the gas provided from the gas unit 200 is injected into the lumen unit 100, the lumen unit 100 may expand. For example, the gas unit 200 may provide the gas to an internal channel IC of the lumen unit 100.

The drug solution unit 300 may provide the drug solution to the lumen unit 100. When the drug solution provided from the drug solution unit 300 is injected into the lumen unit 100, the lumen unit 100 may form an expanded shape. For example, the drug solution unit 300 may provide the drug solution to an external channel EC of the lumen unit 100. The lumen unit 100 may include a first lumen unit 110 formed of an inner coating 130 and a second lumen unit 120 formed of an outer coating 160. Each of the first lumen unit 110 and the second lumen unit 120 may form a balloon shape.

For example, the lumen unit 100 may be expanded by the injected gas or/and drug solution. If the gas or/and the drug solution injected into the lumen unit 100 is discharged in a state where the lumen unit 100 is expanded, the lumen unit 100 may be contracted. For example, shapes of the inner coating 130 and the outer coating 160 of the lumen unit 100 may be freely changed.

The first lumen unit 110 may be disposed inside the second lumen unit 120. In other words, the inner coating 130 may be disposed inside the outer coating 160. That is, the outer coating 160 may surround the inner coating 130. For example, the inner coating 130 may be accommodated in the outer coating 160.

The inner coating 130 may include a first inner surface 131 and a second inner surface 132 disposed opposite the first inner surface 131. The inner coating 130 may form the internal channel IC. For example, the first inner surface 131 of the inner coating 130 may face the internal channel IC. The internal channel IC may be connected to the gas unit 200.

The inner coating 130 may form a boundary of the internal channel IC. For example, the first inner surface 131 of the inner coating 130 may form the boundary of the internal channel IC.

The outer coating 160 may face the second inner surface 132. The outer coating 160 may include a first outer surface 161 disposed to face the second inner surface 132 and a second outer surface 162 disposed opposite the first outer surface 161.

The external channel EC may be formed between the first outer surface 161 of the outer coating 160 and the second inner surface 132 of the inner coating 130. The external channel EC may be connected to the drug solution unit 300.

A boundary of the external channel EC may be formed by the inner coating 130 and the outer coating 160. For example, the boundary of the external channel EC may be formed by the second inner surface 132 of the inner coating 130 and the first outer surface 161 of the outer coating 160. For example, the second inner surface 132 and the first outer surface 161 may face the external channel EC.

As described above, the drug delivery device 10 according to an embodiment of the present disclosure may have a dual lumen structure including the internal channel IC and the external channel EC. For example, the internal channel IC and the external channel EC may be formed with the inner coating 130 interposed therebetween. That is, the inner coating 130 may form a border between the internal channel IC and the external channel EC.

The internal channel IC and the external channel EC may communicate with each other through a micropore 135 formed in the inner coating 130. The micropore 135 may be a microhole passing through the first inner surface 131 and the second inner surface 132 of the inner coating 130. A plurality of micropores 135 may be provided.

The inner coating 130 may be formed of at least one of polyurethane resin, Teflon resin (Gore-Tex), polycaprolactone (PCL) nanofiber membrane, and mixtures thereof that can form the micropores 135. The polyurethane resin may be a porous polyurethane.

Since the inner coating 130 is not a part that is in direct contact with the affected area AA, the inner coating 130 may not use a biocompatible material. For example, the inner coating 130 may be formed of a material that includes porous micropores, passes through gas such as oxygen and nitrogen, and does not pass through liquid.

The outer coating 160 may be in direct contact with the affected area AA. Accordingly, the outer coating 160 may be formed of a biocompatible material.

Examples of the biocompatible material used for the outer coating 160 may include polyester, polyhydroxyalkanoate (PHAs), poly(α-hydroxy acid), poly(β-hydroxy acid), poly(3-hydroxy butyrate-co-valerate) (PHBV), poly(3-hydroxypropionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), polycaprolactone, polylactide, polyglycolide, poly(lactide-co-glycolide) (PLGA), polydioxanone, polyorthoester, polyether ester, polyanhydride, poly(glycolic acid-co-trimethylene carbonate), polyphosphoester, polyphosphoester urethane, poly(amino acid), polycyanoacrylate, poly(trimethylene carbonate), poly(imino carbonate), poly(tyrosine carbonate), polycarbonate, poly(tyrosine arylate), polyalkylene oxalate, polyphosphazenes, PHA-PEG, ethylene vinyl alcohol copolymer (EVOH), polyurethane, silicone, polyester, polyolefin, polyisobutylene and ethylene-alpha olefin copolymer, styrene-isobutylene-styrene triblock copolymer, acrylic polymer and copolymer, vinyl halide polymer and copolymer, polyvinyl chloride, polyvinyl ether, polyvinyl methyl ether, polyvinylidene halide, polyvinylidene fluoride, polyvinylidene chloride, polyfluoroalkene, polyperfluoroalkene, polyacrylonitrile, polyvinyl ketones, polyvinyl aromatics, polystyrene, polyvinyl ester, polyvinyl acetate, ethylene-methyl methacrylate copolymer, acrylonitrile-styrene copolymer, ABS resin and ethylene-vinyl acetate copolymer, polyamide, alkyd resin, polyoxymethylene, polyimide, polyether, polyacrylate, polymethacrylate, polyacrylic acid-co-maleic acid, chitosan, dextran, cellulose, heparin, hyaluronic acid, alginate, inulin, starch, or glycogen. Specific examples may include at least one selected among polyester, polyhydroxyalkanoate (PHAs), poly(α-hydroxy acid), poly(β-hydroxy acid), poly(3-hydroxy butyrate-co-valerate) (PHBV), poly(3-hydroxypropionate) (PHP), poly(3-hydroxyhexanoate) (PHH), poly(4-hydroxy acid), poly(4-hydroxy butyrate), poly(4-hydroxyvalerate), poly(4-hydroxyhexanoate), poly(ester amide), polycaprolactone, polylactide, polyglycolide, poly(lactide-co-glycolide) (PLGA), polydioxanone, polyorthoester, polyether ester, polyanhydride, poly(glycolic acid-co-trimethylene carbonate), polyphosphoester, polyphosphoester urethane, poly(amino acid), polycyanoacrylate, poly(trimethylene carbonate), poly(imino carbonate), poly(tyrosine carbonate), polycarbonate, poly(tyrosine arylate), polyalkylene oxalate, polyphosphazenes, PHA-PEG, chitosan, dextran, cellulose, heparin, hyaluronic acid, alginate, inulin, starch, or glycogen.

The lumen unit 100 may include the discharge area DA, the delivery area TA, and the injection area IA. In the discharge area DA, a spout part DP through which the gas or the drug solution is discharged may be disposed. In the delivery area TA, a delivery part TP delivering the gas or the drug solution may be disposed. In the injection area IA, a filling part IP filled with the gas or the drug solution may be disposed. Each of the first lumen unit 110 and the second lumen unit 120 may be disposed in the discharge area DA, the delivery area TA, and the injection area IA.

More specifically, the first lumen unit 110 may include a first spout part 112, a first delivery part 115, and a first filling part 118. The second lumen unit 120 may include a second spout part 122, a second delivery part 125, and a second filling part 128.

The first spout part 112 and the second spout part 122 may be disposed in the discharge area DA. Therefore, the spout part DP may include the first spout part 112 and the second spout part 122.

The gas in the discharge area DA may be discharged through the gas discharge port 210. The first spout part 112 disposed in the discharge area DA may cover the gas discharge port 210 and a portion of the gas supply pipe 220 of the gas unit 200. Because of this, the first spout part 112 can prevent the gas from leaking from the first lumen unit 110.

In addition, the first spout part 112 can allow the drug delivery device 10 to control an amount of gas filled in the internal channel IC. That is, a gas pressure of the internal channel IC can be controlled through the first spout part 112.

The drug solution in the discharge area DA may be discharged through the drug solution discharge port 310. The second spout part 122 disposed in the discharge area DA may cover the drug solution discharge port 310 and a portion of the drug solution supply pipe 320 of the drug solution unit 300. Because of this, the second spout part 122 can prevent the drug solution from leaking from the second lumen unit 120. In addition, the second spout part 122 can allow the drug delivery device 10 to easily control an amount of drug solution provided to the external channel EC.

The first spout part 112 may be disposed inside the second spout part 122. For example, the first spout part 112 may be sealed by the second spout part 122. For example, the second spout part 122 may surround the first spout part 112,

As illustrated in FIGS. 1 and 2, the first delivery part 115 and the second delivery part 125 may be disposed in the delivery area TA. The delivery area TA may be connected to the discharge area DA. For example, the delivery area TA may extend from the discharge area DA. For example, the delivery area TA may form a shape extending from the discharge area DA.

The first delivery part 115 and the second delivery part 125 may be disposed in the delivery part TP. The delivery area TA may be a passage through which the gas and the drug solution provided from the gas discharge port 210 and the drug solution discharge port 310 are delivered to the injection area IA.

The first delivery part 115 disposed in the delivery area TA may be a portion of the internal channel IC and may serve to deliver the gas provided from the discharge area DA to the injection area IA. The first delivery part 115 may provide the gas to the external channel EC through the micropores 135 formed in the inner coating 130.

The second delivery part 125 disposed in the delivery area TA may be a portion of the external channel EC. The second delivery part 125 may serve to deliver the drug solution provided from the discharge area DA to the injection area IA.

As illustrated in FIGS. 1 to 3, the first filling part 118 and the second filling part 128 may be disposed in the injection area IA. The injection area IA may be connected to the delivery area TA. For example, the injection area IA may extend from the delivery area TA. For example, the injection area IA may form a shape extending from the delivery area TA.

The first filling part 118 and the second filling part 128 may be disposed in the filling part IP. The injection area IA may be an area that injects a mixed drug solution, that is a mixture of the gas and the drug solution respectively supplied to the first filling part 118 and the second filling part 128, into the affected area AA.

The injection area IA may be filled with the gas or the drug solution, before injecting the mixed drug solution into the affected area AA, to form a pressure of the gas or an injection pressure of the drug solution. The mixed drug solution may be injected into the affected area AA using the gas pressure or the injection pressure formed in the injection area IA.

The first filling part 118 may be filled with the gas delivered from the first delivery part 115. In other words, since the first filling part 118 disposed in the internal channel IC is filled with the gas, the first filling part 118 may expand the inner coating 130. The first filling part 118 may expand the inner coating 130 by the filling of the gas. The first filling part 118 may provide an expansion force to the external channel EC.

The gas positioned in the internal channel IC in the first filling part 118 may move to the external channel EC through the micropores 135 formed in the inner coating 130. For example, the gas positioned in the internal channel IC may move from the first inner surface 131 to the second inner surface 132 of the inner coating 130 through the micropores 135. Therefore, the gas that passes through the micropores 135 may be positioned in the external channel EC.

The gas that passes through the micropores 135 and enters the external channel EC may be formed as micro-gas droplets while contacting the liquid drug solution disposed in the external channel EC. For example, when the gas is oxygen, an oxygen gas may form micro-oxygen droplets in the external channel EC while passing through the micropores 135.

When the drug solution is injected into the external channel EC, the external channel EC may expand due to the pressure of the drug solution. When the external channel EC expands, the outer coating 160 may expand. When the external channel EC expands, the inner coating 130 may expand.

For example, when the drug solution is injected into the external channel EC, a distance between the inner coating 130 and the outer coating 160 may increase. As another example, the distance between the inner coating 130 and the outer coating 160 may increase within a predetermined range.

For example, a spacer may be disposed between the inner coating 130 and the outer coating 160. For example, the spacer may have elasticity. For example, when the external channel EC expands, an elastic force (or restoring force) may be applied between the inner coating 130 and the outer coating 160.

A microneedle plate 165 may be disposed on the second filling part 128 disposed in the injection area IA. For example, the microneedle plate 165 may be coupled to or formed on the outer coating 160 positioned on the second filling part 128.

For example, the microneedle plate 165 may be formed by protruding a portion of the outer coating 160 positioned on the second filling part 128. For example, the microneedle plate 165 may be coupled to the second outer surface 162 of the outer coating 160.

The microneedle plate 165 may be in direct contact with the affected area AA. The microneedle plate 165 may include a microneedle 166. The microneedle 166 may form a shape protruding from the second outer surface 162 of the outer coating 160. A distal end of the microneedle 166 may form a tip portion.

For example, the microneedle 166 may form an overall horn shape. For example, the microneedle 166 may form an overall cone shape.

The microneedle plate 165 may push the microneedle 166 into the inside of the affected area AA. For example, when the microneedle 166 enters the affected area AA, a first microhole 168 (see FIG. 6) may be in contact with the affected area AA. Therefore, the microneedle plate 165 can inject the drug solution and micro-oxygen droplets directly into the affected area AA.

As described above, the drug delivery device 10 according to the present disclosure has the dual lumen structure including the internal channel IC and the external channel EC, and thus can push the gas and the drug solution into the inside of the affected area AA using the gas pressure.

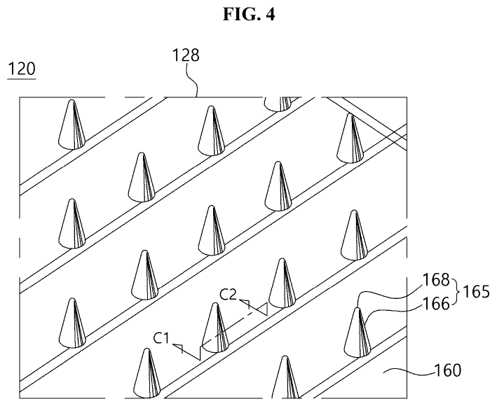

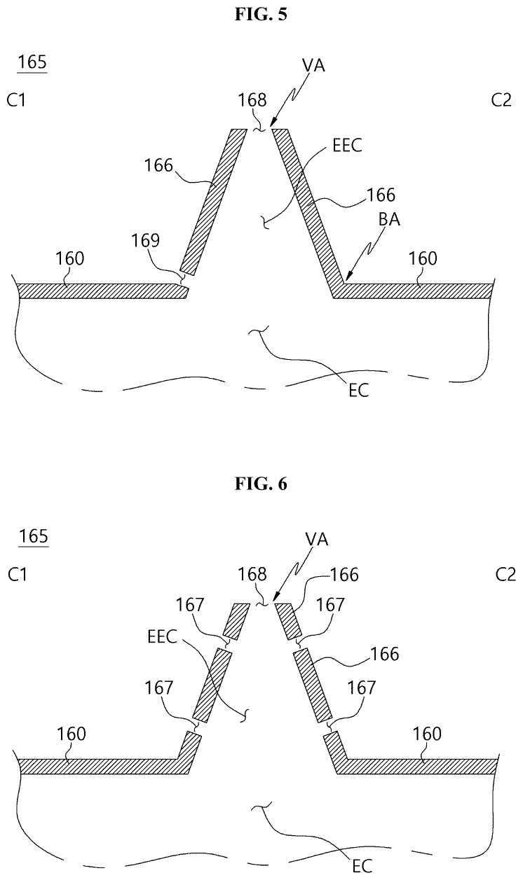

FIG. 4 is a partial perspective view of an outer coating according to an embodiment of the present disclosure. FIG. 5 is a cross-sectional view taken along C1-C2 of FIG. 4. FIG. 6 is a cross-sectional view of a microneedle plate according to another embodiment of the present disclosure.

FIGS. 4 to 6 will be described by referring to FIGS. 1 to 3 to avoid redundant description and for easy explanation.

Referring to FIGS. 4 to 6, the drug delivery device 10 according to an embodiment of the present disclosure may include the second lumen unit 120 in direct contact with the affected area AA of the patient. The second filling part 128 may be disposed in the second lumen unit 120.

The microneedle plate 165 may include a plurality of microneedles 166 and a plurality of microholes 168 and 169 that communicate the external channel EC with an outside. At least one microhole is disposed in each of the plurality of microneedles 166. Here, the outside may be the affected area AA.

The microneedle 166 may be disposed to protrude in a cone shape from the surface of the outer coating 160. This embodiment describes the microneedle 166 of the cone shape, but can use the microneedle 166 of any shape that protrudes outward, such as a needle shape and a hook shape.

Therefore, the microneedle plate 165 can easily push the microneedle 166 into the inside of the affected area AA. The microneedle plate 165 can easily bring the surface of the outer coating 160 disposed on the second filling part 128 into close contact with the affected area AA.

The microneedle 166 protruding from the second outer surface 162 of the outer coating 160 may be formed in a horn shape. A space may be formed inside the microneedle 166. The space formed in the microneedle 166 may communicate with the external channel EC.

In other words, the space formed in the microneedle 166 may be formed by extending from the external channel EC. The space formed in the microneedle 166 may be referred to as an extension channel EEC. The extension channel EEC may be formed by extending from the external channel EC.

In other words, the extension channel EEC may be formed in a space formed as the microneedle 166 protrudes. The extension channel EEC may protrude further outward than the external channel EC.

A plurality of microholes 167, 168, and 169 may be disposed in a portion of the microneedle 166 to communicate between the external channel EC and the affected area AA. The microholes 167, 168, and 169 may serve as a passage for delivering a mixed drug solution, which is a mixture of the drug solution and the micro-gas droplets disposed in the external channel EC, directly to the affected area AA.

The microneedle 166 may form the overall horn shape. For example, a tip portion of the microneedle 166 may be referred to as a vertex area VA. For example, a portion of the bottom of the microneedle 166 connected to the outer coating 160 may be referred to as a bent area BA.

The microneedle plate 165 may include the microholes 167, 168, and 169. The microholes 167, 168, and 169 may be openings or holes formed in the microneedle 166. The microholes 167, 168, and 169 may be connected to or communicate with the extension channel EEC.

For example, the microholes 167, 168, and 169 may include a second microhole 169 formed or positioned in the bent area BA. For example, the microholes 167, 168, and 169 may include a first microhole 168 formed or positioned in the vertex area VA. For example, the microholes 167, 168, and 169 may include a third microhole 167 formed in the side of the microneedle 166. The third microhole 167 may be disposed between the vertex area VA and the bent area BA.

The extension channel EEC may be disposed between the external channel EC and the affected area AA. The microholes 168 and 169 may allow the extension channel EEC to communicate with the affected area AA.

The second microhole 169 may be disposed adjacent to the second outer surface 162 of the outer coating 160. Therefore, the second microhole 169 may be disposed in the epidermis of the affected area AA. Hence, the second microhole 169 may be a passage that receives the gas or the drug solution from the external channel EC and the extension channel EEC and provides a target amount of gas or drug solution to the affected area AA. The second microhole 169 may be a passage for providing the mixed drug solution to the affected area AA even if a gas pressure or a drug solution injection pressure provided to the extension channel EEC is reduced.

The microneedle 166 may include at least one of the first microhole 168 and the second microhole 169. It is possible to easily control an amount of gas and an amount of drug solution by disposing at least one of the first microhole 168 and the second microhole 169 in the microneedle 166.

Referring to FIG. 6, the microneedle 166 may include the third microhole 167. At least one third microhole 167 may be disposed in a side portion of the microneedle 166. The side portion of the microneedle 166 may indicate a portion between the vertex area VA and the bent area BA of the microneedle 166.

The third microhole 167 may face the affected area AA. Therefore, the third microhole 167 may be a passage through which the drug solution or the gas is provided to the inside of the affected area AA.

A plurality of third microholes 167 may be provided. For example, the plurality of third microholes 167 may be sequentially arranged from the bent area BA toward the vertex area VA of the microneedle 166. As a result, the drug solution and/or the gas can be delivered evenly to the affected area AA.

The microneedle 166 may include at least one of the first microhole 168, the second microhole 169, and the third microhole 167. The amount of gas and the amount of drug solution can be easily controlled by arranging at least one of the first microhole 168, the second microhole 169, and the third microhole 167 in the microneedle 166. In the present disclosure, the number of microholes 167, 168, and 169 may be adjusted considering a strength of the microneedle 166.

As described above, the drug delivery device 10 according to an embodiment of the present disclosure includes the microneedles 166 penetrating the inside of the affected area AA, and arranges at least one microhole (167, 168, and 169) in the microneedles 166 to thereby inject the gas or the drug solution into the inner area of the affected area AA.

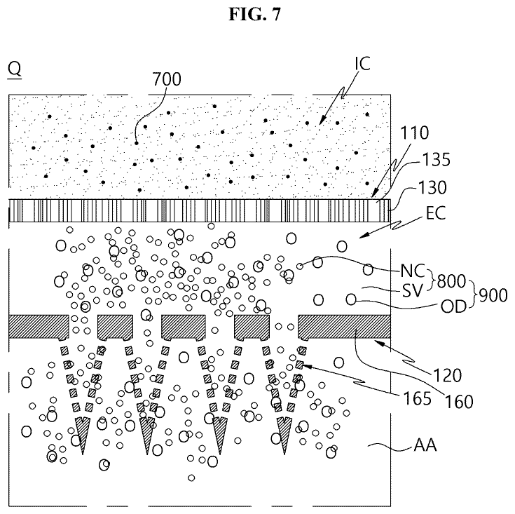

FIG. 7 is an enlarged cross-sectional view of a portion “Q” of FIG. 1.

FIG. 7 will be described by referring to FIGS. 1 to 6 to avoid redundant description and for easy explanation.

Referring to FIG. 7, the drug delivery device 10 may form a mixed drug solution 900, that is a mixture of a drug solution 800 and a gas 700, in the injection area IA.

The gas 700 may be, for example, oxygen gas (O₂). Hereinafter, the gas is referred to as oxygen gas. The oxygen gas 700 may be disposed in the internal channel IC.

Due to the gas pressure, the oxygen gas 700 may pass through the micropores 135 formed in the inner coating 130. A size of the micropore 135 may be 1 nm to 50 nm.

If the size of the micropore 135 is larger than 50 nm, a particle size of a nanoparticle composite NC may be 50 nm to 200 nm. Because of this, the nanoparticle composite NC may move from the external channel EC to the internal channel IC.

If the nanoparticle composite NC moves from the external channel EC to the internal channel IC, it may be difficult to deliver the nanoparticle composite to the affected area AA. Therefore, the size of the micropore 135 may be 50 nm or less.

The oxygen gas 700 may be provided to the internal channel IC at a gas pressure of 1 atm to 5 atm. A minimum pressure of the gas required for the gas to pass through the micropores 135 may be 1 atm. 1 atm may be an atmospheric pressure. In addition, an oxygen hyperbaric chamber for treatment may provide the oxygen gas 700 with a pressure of 5 atm or less. If the pressure of the oxygen gas 700 exceeds 5 atm, the excessive gas may be provided to the affected area AA.

In other words, due to the gas pressure, the oxygen gas 700 may pass through the micropores 135 and move from the internal channel IC to the external channel EC. The oxygen gas 700 that has passed through the micropores 135 may form micro-oxygen droplets OD while contacting the drug solution 800 disposed in the external channel EC.

The micro-oxygen droplets OD may be mixed with the drug solution 800 disposed in the external channel EC to form the mixed drug solution 900. The mixed drug solution 900 may be diffused into not only the external channel EC but also the extension channel EEC. The mixed drug solution 900 diffused into the extension channel EEC may pass through the microholes 167, 168, and 169 under the gas pressure and the pressure, at which the drug solution 800 is injected, and may be delivered to the affected area AA.

The mixed drug solution 900 formed by mixing the micro-oxygen droplets OD and the drug solution 800 may pass through the microholes 167, 168, and 169 more effectively than the drug solution 800. For example, when the drug solution 800 passes through the microholes 167, 168, and 169, the microholes 167, 168, and 169 may be blocked by the drug solution 800 due to viscosity of the drug solution 800.

The mixed drug solution 900 itself may contain a large amount of oxygen. As a result, the mixed drug solution 900 can act more effectively on cancer types that are resistant to hypoxia, thereby inducing an increase in an anticancer response rate.

The drug solution 800 is a drug delivered to tumor tissue such as cancer tissue and may use the nanoparticle composite NC mediated by nanoparticles.

For example, the drug solution 800 may include the nanoparticle composite NC mixed in a solvent SV. Here, the solvent SV may dissolve the nanoparticle composite NC or surround the nanoparticle composite NC. The solvent SV and the nanoparticle composite NC may be used variously depending on types of affected area. Accordingly, the solvent SV and the nanoparticle composite NC are not specified.

The solvent SV and the nanoparticle composite NC may be disposed in the external channel EC. The solvent SV and the nanoparticle composite NC may also be disposed in the extension channel EEC. The nanoparticle composite NC may have a particle size of 50 nm to 200 nm.

If the particle size of the nanoparticle composite NC is less than 50 nm, it may be difficult to manufacture the nanoparticles. Additionally, considering a relationship with the size of the micropores 135, the particle size of the nanoparticle composite NC may be 50 nm or more. Further, if the particle size of the nanoparticle composite NC exceeds 200 nm, it may be difficult to accumulate the nanoparticle composite NC in the tumor tissue of the affected area.

For example, the tumor tissue may be in a mutated state and may be formed into a chaotic shape, which lead to formation of gaps in the tumor tissue. The gap of the tumor tissue may be formed to be about 200 nm. Therefore, when the particle size of the nanoparticle composite NC is 200 nm or less, the nanoparticle composite NC can be easily accumulated in the gaps of the tumor tissue.

In the drug delivery device 10, the nanoparticle composite NC may use a material that reacts to radiation, ultrasound, magnetic field, etc. In other words, the nanoparticle composite NC may allow the tumor tissue to react to radiation, ultrasound, magnetic field, etc. to induce a tumor microenvironment suitable for anticancer treatment (mechanotransduction).

For example, when the nanoparticle composite NC uses a material reacting to the ultrasound, micro-distribution diffusion may occur at the same time as anticancer treatment effects such as sono-dynamic therapy (SDT) and local heating. Furthermore, through the micro-distribution diffusion, treatment on a per cell basis is possible, and an amount of drug solution 800 used can be reduced. The treatment on a per cell basis and the reduction in the amount of drug solution 800 used can reduce a treatment burden on patients.

As described above, the mixed drug solution 900 which is a mixture of the micro-oxygen droplets OD and the drug solution 800 may be formed in the external channel EC. The mixed drug solution 900 may move to the extension channel EEC by a gas pressure of the oxygen gas and an injection pressure of the drug solution 800. The mixed drug solution 900 disposed in the extension channel EEC may be injected into the affected area AA through the provided pressures.

As described above, the drug delivery device 10 according to the present disclosure delivers the drug solution 800 including the nanoparticle composite NC and the oxygen gas 700 through the dual lumen structure and then forms the mixed drug solution 900, which is a mixture of the drug solution 800 and the oxygen gas 700, by a pressure of gas introduced through the micropores 135 of the inner coating 130, thereby overcoming hypoxia in the affected area AA and maximizing anticancer drugs or radiation treatment.

Referring to FIGS. 1 to 7, the lumen unit 100 may be flexible and/or elastic. For example, the lumen unit 100 may be formed of a material including a flexible material or an elastic material.

For example, when gas is injected into the first lumen unit 110, the first lumen unit 110 may expand. For example, when the gas 700 is injected into the first lumen unit 110, the first lumen unit 110 may form an elastic force. Due to the elastic force formed by the first lumen unit 110, a pressure of the gas 700 injected into the first lumen unit 110 may increase.

When the pressure of the gas 700 injected into the first lumen unit 110 increases, the gas 700 may pass through the micropores 135 and may be introduced into the external channel EC. While the gas 700 passes through the micropores 135, the gas 700 may change into microbubbles in the external channel EC. For example, if the gas 700 is an oxygen molecule, the gas 700 may change into the micro-oxygen droplets OD while the gas 700 passes through the micropores 135.

The micro-oxygen droplets OD and the drug solution 800 may be mixed in the external channel EC to form the mixed drug solution 900. The mixed drug solution 900 may refer to the drug solution 800 with which the micro-oxygen droplets OD are combined.

When the first lumen unit 110 expands, the first lumen unit 110 may transfer a pressure to the second lumen unit 120. That is, when the gas 700 is injected into the first lumen unit 110 and the first lumen unit 110 expands, a pressure of the external channel EC may increase as the first lumen unit 110 pushes the second lumen unit 120. When the pressure of the external channel EC increases, the mixed drug solution 900 may be discharged to the outside through the microholes 167, 168, and 169. That is, the mixed drug solution 900 may be applied to the affected area AA.

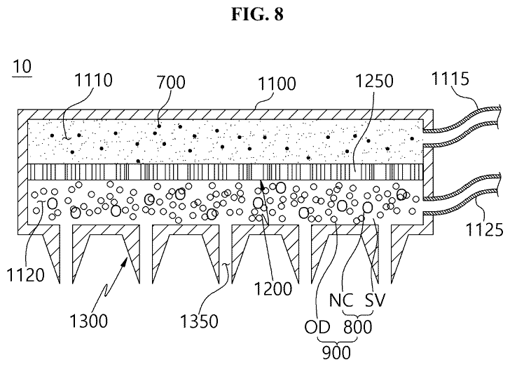

FIG. 8 is a cross-sectional view illustrating a drug delivery device including a flat lumen unit.

Referring to FIG. 8, the drug delivery device 10 may include a flat lumen unit 1100. The flat lumen unit 1100 may form a shape of a plate with a thickness. The flat lumen unit 1100 may form a space therein.

The drug delivery device 10 may include an inner coating 1200. The inner coating 1200 may be positioned or installed inside the flat lumen unit 1100. For example, the inner coating 1200 may divide the inner space of the flat lumen part 1100 into two part. For example, the inner coating 1200 may divide the inner space of the flat lumen part 1100 into an upper part and a lower part.

For example, the drug delivery device 10 may include a first channel 1110 and a second channel 1120. The first channel 1110 may be a space formed by one surface (e.g., upper surface) of the flat lumen part 1100 and the inner coating 1200. The second channel 1120 may be a space formed by other surface (e.g., lower surface) of the flat lumen unit 1100 and the inner coating 1200.

The drug delivery device 10 may include micropores 1250. The micropores 1250 may refer to pores formed in the inner coating 1200. For example, the micropores 1250 may allow molecules in a gas state to pass through. For example, the micropores 1250 may prevent molecules in a liquid state from passing through.

The drug delivery device 10 may include microneedles 1300. The microneedle 1300 may be connected to, coupled to, communicate with, fixed to, or formed on the flat lumen unit 1100. For example, the microneedle 1300 may be connected to or communicate with the second channel 1120.

The drug delivery device 10 may include microholes 1350. The microhole 1350 may be a hole formed in the microneedle 1300. The microhole 1350 may be connected to or communicate with the second channel 1120.

The drug delivery device 10 may include a gas hose 1115. The gas hose 1115 may be connected to or communicate with the first channel 1110. The gas 700 may be injected into the first channel 1110 through the gas hose 1115. The gas 700 may be discharged from the first channel 1110 to the outside through the gas hose 1115.

The drug delivery device 10 may include a drug hose 1125. The drug hose 1125 may be connected to or communicate with the second channel 1120. The drug solution 800 may be injected into the second channel 1120 through the drug hose 1125. The drug solution 800 may be discharged from the second channel 1120 to the outside through the drug hose 1125.

In a state in which the drug solution 800 is positioned in the second channel 1120, the gas 700 may be injected into the first channel 1110. When the gas 700 is injected into the first channel 1110, a pressure in the first channel 1110 may increase. When the pressure increases in the first channel 1110, the gas 700 positioned in the first channel 1110 may pass through the micropores 1250 and may be delivered to the second channel 1120.

While the gas 700 passes through the micropores 1250, the gas 700 may form micro-gas droplets. For example, if the gas 700 is an oxygen molecule, the gas 700 may form micro-oxygen droplets OD while passing through the micropores 1250.

The micro-oxygen droplets OD may be mixed in the second channel 1120 to form the mixed drug solution 900. The mixed drug solution 900 may refer to the drug solution 800 with which the micro-oxygen droplets OD are combined.

When the gas 700 is introduced into the second channel 1120, a pressure of the second channel 1120 may increase. When the pressure of the second channel 1120 increases, the mixed drug solution 900 may be discharged to the outside. That is, the mixed drug solution 900 may be applied to the affected area AA (see FIG. 7).

Some embodiments or other embodiments of the present disclosure described above are not mutually exclusive or distinct from each other. Configurations or functions of some embodiments or other embodiments of the present disclosure described above can be used together or combined with each other.

It is apparent to those skilled in the art that the present disclosure can be embodied in other specific forms without departing from the spirit and essential features of the present disclosure. Accordingly, the above detailed description should not be construed as limiting in all aspects and should be considered as illustrative. The scope of the present disclosure should be determined by rational interpretation of the appended claims, and all modifications within an equivalent scope of the present disclosure are included in the scope of the present disclosure.

1. A drug delivery device comprising:
a lumen unit configured to form an internal channel and an external channel;
a gas unit configured to supply a gas to the internal channel; and
a drug solution unit configured to supply a drug solution to the external channel,
wherein the lumen unit includes:
an inner coating including a first inner surface, a second inner surface positioned opposite the first inner surface, and micropores passing through the first inner surface and the second inner surface;
an outer coating including a first outer surface facing the second inner surface, a second outer surface positioned opposite the first outer surface, and a microneedle plate formed on a surface of the second outer surface;
the internal channel surrounded by the first inner surface; and
the external channel formed between the second inner surface and the first outer surface,
wherein the gas and the drug solution are mixed in the external channel to form a mixed drug solution, and
wherein the mixed drug solution is supplied to an outside through the microneedle plate.

2. The drug delivery device of claim 1, wherein the internal channel and the external channel communicate with each other through the micropores.

3. The drug delivery device of claim 1, wherein the micropores allow the gas to pass through from the internal channel to the external channel, and
wherein the micropores prevent the drug solution from passing through from the external channel to the internal channel.

4. The drug delivery device of claim 1, wherein the inner coating is formed of at least one of polyurethane resin, Teflon resin, polycaprolactone (PCL) nanofiber membrane, and mixtures thereof.

5. The drug delivery device of claim 1, wherein the outer coating includes polyester, polyhydroxyalkanoate (PHAs), poly(α-hydroxy acid), poly(β-hydroxy acid), poly(3-hydroxybutyrate-co-valerate) (PHBV), poly(3-hydroxypropionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), polycaprolactone, polylactide, polyglycolide, poly(lactide-co-glycolide) (PLGA), polydioxanone, polyorthoester, polyether ester, polyanhydride, poly(glycolic acid-co-trimethylene carbonate), polyphosphoester, polyphosphoester urethane, poly(amino acid), polycyanoacrylate, poly(trimethylene carbonate), poly(imino carbonate), poly(tyrosine carbonate), polycarbonate, poly(tyrosine arylate), polyalkylene oxalate, polyphosphazenes, PHA-PEG, ethylene vinyl alcohol copolymer (EVOH), polyurethane, silicone, polyester, polyolefin, polyisobutylene and ethylene-alpha olefin copolymer, styrene-isobutylene-styrene triblock copolymer, acrylic polymer and copolymer, vinyl halide polymer and copolymer, polyvinyl chloride, polyvinyl ether, polyvinyl methyl ether, polyvinylidene halide, polyvinylidene fluoride, polyvinylidene chloride, polyfluoroalkene, polyperfluoroalkene, polyacrylonitrile, polyvinyl ketones, polyvinyl aromatics, polystyrene, polyvinyl ester, polyvinyl acetate, ethylene-methyl methacrylate copolymer, acrylonitrile-styrene copolymer, ABS resin and ethylene-vinyl acetate copolymer, polyamide, alkyd resin, polyoxymethylene, polyimide, polyether, polyacrylate, polymethacrylate, polyacrylic acid-co-maleic acid, chitosan, dextran, cellulose, heparin, hyaluronic acid, alginate, inulin, starch, or glycogen, and
wherein the outer coating is specifically formed of at least one selected among polyester, polyhydroxyalkanoate (PHAs), poly(α-hydroxy acid), poly(β-hydroxy acid), poly(3-hydroxybutyrate-co-valerate) (PHBV), poly(3-hydroxypropionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), polycaprolactone, polylactide, polyglycolide, poly(lactide-co-glycolide) (PLGA), polydioxanone, polyorthoester, polyether ester, polyanhydride, poly(glycolic acid-co-trimethylene carbonate), polyphosphoester, polyphosphoester urethane, poly(amino acid), polycyanoacrylate, poly(trimethylene carbonate), poly(imino carbonate), poly(tyrosine carbonate), polycarbonate, poly(tyrosine arylate), polyalkylene oxalate, polyphosphazenes, PHA-PEG, chitosan, dextran, cellulose, heparin, hyaluronic acid, alginate, inulin, starch, or glycogen.

6. The drug delivery device of claim 1, wherein the lumen unit includes;
a discharge area in which a spout part discharging the gas or the drug solution is disposed;
a delivery area in which a delivery part delivering the gas or the drug solution is disposed; and
an injection area in which a filling part filled with the gas or the drug solution is disposed,
wherein the lumen unit further includes;
a first lumen unit formed of the inner coating; and
a second lumen unit formed of the outer coating,
wherein the first lumen unit and the second lumen unit are disposed in the discharge area, the delivery area, and the injection area.

7. The drug delivery device of claim 6, wherein the first lumen unit includes:
a first spout part configured to discharge the gas and disposed in the discharge area;
a first delivery part configured to deliver the gas and disposed in the delivery area; and
a first filling part filled with the gas and disposed in the injection area.

8. The drug delivery device of claim 6, wherein the second lumen unit includes:
a second spout part configured to discharge the drug solution and disposed in the discharge area;
a second delivery part configured to deliver the drug solution and disposed in the delivery area; and
a second filling part filled with the drug solution and disposed in the injection area.

9. The drug delivery device of claim 8, wherein the microneedle plate is disposed on the second filling part disposed in the injection area and is formed by protruding a portion of a surface of the outer coating.

10. The drug delivery device of claim 1, wherein the microneedle plate includes:
a plurality of microneedles; and
a plurality of microholes configured to communicate the external channel with the outside, at least one microhole being disposed in each of the plurality of microneedles.

11. The drug delivery device of claim 10, wherein the microneedle plate pushes the plurality of microneedles into an inside of an affected area and brings a surface of the outer coating into close contact with a surface of the affected area.

12. The drug delivery device of claim 10, wherein an extension channel extending from the external channel is formed inside the microneedle.

13. The drug delivery device of claim 12, wherein the microholes communicate the external channel with the outside.

14. The drug delivery device of claim 10, wherein the microholes include at least one of:
a first microhole in which a tip of the microneedle is formed at a vertex of a sharp end;
a second microhole formed in a bent area in which the outer coating is bent to form the microneedle; and
a third microhole formed in a side of the microneedle disposed between the vertex and the bent area.

15. The drug delivery device of claim 1, wherein a size of each of the micropores is 1 nm to 50 nm.

16. The drug delivery device of claim 1, wherein when the gas is an oxygen gas, the gas passes through the micropores and is converted into micro-oxygen droplets.

17. The drug delivery device of claim 1, wherein a pressure of the gas provided to the internal channel is 1 atm to 5 atm.

18. The drug delivery device of claim 1, wherein the drug solution is a nanoparticle composite that is a mixture of a solvent and nanoparticles.

19. The drug delivery device of claim 18, wherein a particle size of the nanoparticle composite is 50 nm to 200 nm.

20. The drug delivery device of claim 1, wherein the lumen unit is flexible and elastic.
